(19) United States
(12) Patent Application Publication
(10) Pub. No.: US 2023/0053636 A1
(43) Pub. Date: Feb. 23, 2023

(54) ECOLOGICAL SUPPORT FOR COSMETIC PRODUCTS AND RELATED FORMATION PROCESS

(71) Applicant: INTERCOS S.p.A., Milano (IT)

(72) Inventors: Ibrahim HANNO, GORGONZOLA (MI) (IT); Donato SMALDONE, PARABIAGO (MI) (IT); Gabriele DEPTA, MONZA (MB) (IT); Sara BETTINELLI, PARABIAGO (MI) (IT)

(73) Assignee: INTERCOS S.p.A., Milano (IT)

(21) Appl. No.: 17/796,060

(22) PCT Filed: Jan. 27, 2021
(86) PCT No.: PCT/EP2021/051851

§ 371 Date: Jul. 28, 2022

(30) Foreign Application Priority Data
Jan. 29, 2020 (IT) 102020000001678

(51) Int. Cl. A61K 8/02; A61K 8/36; A61K 8/73; A61K 8/64; A61K 8/60; A61K 8/26; A61Q 1/12; A61Q 1/02

(52) U.S. Cl. CPC A61K 8/0216; A61K 8/26; A61K 8/36; A61K 8/60; A61K 8/645; A61K 8/731; A61K 8/732; A61Q 1/02; A61Q 1/12; A61K 2800/524; A61K 2800/805

(57) ABSTRACT

There is described the manufacturing of a support (6) for cosmetic products, which consists of a dry and solidified set of a mixture consisting of non-synthetic ingredients of natural origin only. The support can be manufactured so as to form a base for supporting and anchoring a cosmetic product inside a container (9) or a biodegradable container in the shape of a rigid and self-supporting cosmetic wafer for anhydrous or gel cosmetic products.

The present invention relates to an ecological support for cosmetic products and to a related formation process.

Plastic consumption has risen from 15 million tons in 1964 to more than 310 million tons today. Plastic waste now pollutes a large part of natural environments. Some studies estimate that there are up to 150 tons of plastic in the oceans and that if the production trend continues in the current manner, plastic could reach 34 billion tons by 2050, at least 12 tons of which would be waste scattered throughout the environments. These figures suggest how fundamental the recycling or replacement of plastic objects is, with the aim of achieving a radical decrease in the production of this material.

The possibility to replace plastic both as an ingredient and as primary and secondary packaging is a process underway in all industrial and nonindustrial areas. In cosmetics, the replacement of raw materials which generate microplastics is already a process which has been underway for some time, with first significant results.

EP 1078625 A2 describes a solid or viscous cosmetic composition enclosed inside a sheath containing solid proteins, liquid or solution polyols, solid polyols, oils, and emollients. This solution has the drawback that the liquid and/or oily part tends to partially mix with the cosmetic composition over time, thus contaminating it and modifying the properties and cosmetic effects thereof.

It is the general object of the present invention to successfully replace the current plastic supports for cosmetic products with supports made of other material which do not modify the cosmetic properties of the supported cosmetic product in any manner.

In particular, it is object of the present invention to manufacture differently and with a different material the so-called anchoring grids usually used in large volumes (about 2 million pieces per month, therefore with high consumption of plastic) for packaging cosmetic products.

It is another object to manufacture a biodegradable container for any type of cosmetic product.

In order to achieve the aforesaid objects, the present invention suggests a support for cosmetic products as defined in claim 1.

The exercise carried out was to transfer the use of non-synthetic ingredients of natural origin, already used for some time in the food industry, as alternative packaging to polystyrene or plastic from food technology to cosmetics.

Among the possible uses of the ecological support formed from the natural ingredients described above, that related to anchoring grids for cosmetic products is of particular importance.

Such grids are normally made of plastic material and have the task of anchoring themselves to the cosmetic product layer to form the support thereof for the packaging inside a suitable package or box.

It has now been seen that it is possible to replace such grids made of plastic material with corresponding supports made of a material of natural origin, also referred to as “natural grids”, by pouring an aqueous fluid mixture consisting of natural, non-synthetic ingredients according to the invention above a cosmetic product layer, in powder or fluid state, previously introduced into a mold and passing the whole in an oven at 60° C., where the aqueous mixture dries and becomes rigid, irreversibly adhering to the cosmetic product layer.

The process according to the invention is defined in claim11.

It was noted that:
1. The natural grid firmly attaches to all types of cosmetic products.
2. Whether the product has been gelled for a few minutes or after 1 day, therefore cold by now, there are no “sticking” defects.
3. The mixture of the natural grid is cold processed, not containing raw materials which are to be activated with temperature; therefore, no energy costs are added to the process.
4. The natural grid mixture is fluid and therefore spreads easily over the surface of the cosmetic product.
5. The structure of the natural grid is obtained by drying it in a stove or oven at temperatures above 50° C.
6. From a production point of view, it is no longer necessary to use an operator to insert the plastic grid, as the mixture of the natural grid, being fluid, is dosed directly by a volumetric dosing device.
7. It occurs that the plastic grid traces the shape thereof on the main face of the product. This defect disappears with the natural grid.
8. After drying, the product is extracted simply by overturning the mold.
9. The production costs of the molds are eliminated when new formats must be created.
10. The dry substrate is compatible with hot glues.
11. At the production level, the preparation of the mixture with which the natural grid is obtained can be carried out in a single workstation, in large amounts, and then distributed on the individual lines. Each machine needs to be provided with a simple volumetric dosing device.
12. The natural cosmetic support passes the tests which are performed before the product is marketed (tilting and dripping).

The application features of the natural grid are comparable to those of plastics, with some additional advantages:
it gives a geometric shape to the cosmetic product;
it allows manipulation during the packaging;
it allows gluing the cosmetic product to the primary packaging, as the plastic grid does;
it remains firmly glued to the packaging, allowing it to pass quality control tests;
it does not undergo changes in the shape or structure thereof over time;
it is an inert material, which does not transfer any of the ingredients thereof to the cosmetic product;
it allows a “plastic free” declaration when the primary packaging becomes a cardboard or biodegradable case.

In order to form the aforesaid natural grid, the mixture used preferably includes the following formulation:

Table 1

| | |
|---|---|
| Ingredients | Percentages by weight |
| water | from 20.00% to 80.00% |
| organic acids | from 0.05% to 2.00% |
| cellulose or derivatives | from 1.00% to 10.00% |
| vegetal proteins | from 0.10% to 20.00% |
| sugars | from 1.00% to 20.00% |
| starches or dextrins | from 10.00% to 70.00% |
| mica | from 10.00% to 20.00% |
| preservatives | from 0.20% to 1.00% |

and even more preferably the formulation indicated in the following table:

Table 2

| Ingredients | Percentages by weight |
|---|---|
| water | 59.20% |
| organic acids | 0.20% |
| cellulose or derivatives | 1.00% |
| vegetal proteins | 1.00% |
| sugars | 8.10% |
| starches or dextrins | 15.50% |
| mica | 14.00% |
| preservatives | 1.00% |

The formula starts from a food wafer recipe, in which the flour has been replaced by natural mica and the protein part thereof (gluten) has been replaced by vegetal proteins (pea, lentil, bean, broad bean, etc.), now easily available in the market of ingredients for cosmetic products.

The bearing structure is ensured by, in addition to proteins, cellulose or hydrocolloids derived from cellulose (crystalline cellulose, methylcellulose, hydroxypropyl cellulose, hydroxypropyl methylcellulose, carboxymethyl cellulose). These hydrocolloids are activated in water under strong stirring until a foam is obtained.

The sugars present in the formula are the third key ingredient which allows stabilizing the foam created during the preparation of the mixture and after drying. They make the structure very rigid and capable of being perfectly anchored to the cosmetic product to be supported. These sugars can be monosaccharides, disaccharides, or polysaccharides of vegetable origin (maize, rice, tapioca, wheat, or legumes). The origin thereof can be starch which is dextrinized enzymatically.

Starch as it is or modified is used as a filling agent in combination with natural mica. Mica gives the substrate a white color and controls the absorption of water.

Citric acid, sorbic acid, benzoic acid, and hydrochloric acid are preferably used as organic acids.

Preservatives, for example caprylyl glycol, hexylene glycol, ethylhexyl glicerine, ethylparaben, methylparaben, propylparaben, phenoxyethanol, sodium benzoate, potassium sorbate and/or sodium dehydroxyacetate, are used since the formula contains a high amount of water, which is to be preserved.

Moreover, with the same concept it is possible to obtain several other types of ecological supports for cosmetic products.

In particular, it is possible to obtain a biodegradable, rigid and self-supporting container for anhydrous or gelled cosmetic products, which then need to be dried.

In this case, a cosmetic wafer is obtained comprising the following ingredients:
non-allergenic but texturizing vegetable proteins
rice starches of the type used in lipsticks
maltodextrins and/or sugars
hydroxypropyl cellulose
cosmetic oil
hydrogenated lecithin powder
mica- and/or talc-based filler
hydrocolloids
water

The vegetal proteins used are raw materials which are readily available and of relatively low cost.

Maltodextrins and sugars in general, in combination with hydroxypropyl cellulose, allow obtaining excellent rigidity and reducing fragility.

The amount of water must be such as to determine a liquid/creamy mixture which can be poured into a cooking mold.

Once the mixture has been prepared with the aforesaid ingredients, the mixture itself is poured into a mold which is suitable for defining a shell-shaped product, which is then inserted into a wafer oven with coupled walls mechanically moved by motors.

During cooking, the water evaporates almost completely, leaving a product which has about 1-2% humidity.

The hydrocolloids used, of any type, allow managing the internal porosity of the support structure after cooking. Without hydrocolloids, the structure would remain very open and more fragile.

By way of example, a process for manufacturing a natural grid support for a cosmetic product will be described in greater detail below.

For the description of the process, reference will be made to the accompanying drawings, in which:

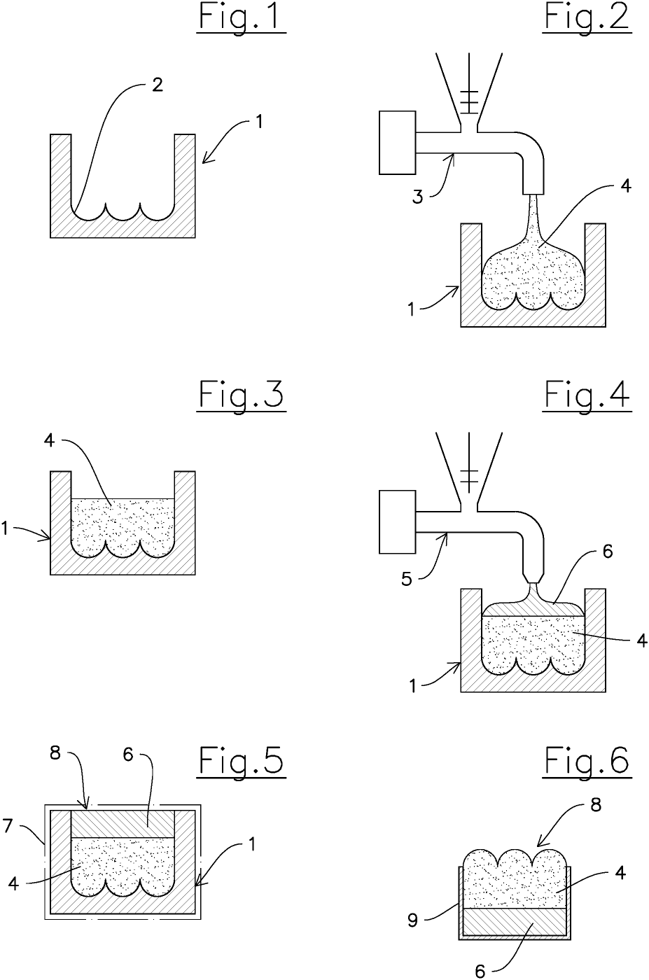

FIG. 1 shows the mold before filling;

FIG. 2 shows the step of filling the mold with a cosmetic product;

FIG. 3 shows the mold filled with a layer of cosmetic product;

FIG. 4 shows the step of pouring a previously prepared aqueous fluid mixture onto the layer of cosmetic product;

FIG. 5 shows the mold and the contents thereof inside a drying and solidifying oven;

FIG. 6 shows the solidified set formed by the cosmetic product and the support base or grid of natural ingredients, overturned and placed inside a container.

The process includes, as a preparatory step, the creation of an aqueous fluid mixture consisting only of non-synthetic ingredients of natural origin, preferably those indicated in the previous table 1, even more preferably with the percentages indicated in table 2.

An appropriate mold is then selected, such as that indicated by reference numeral 1 as a whole in FIG. 1, which has the inner bottom wall 2 shaped complementarily to the desired aesthetic shape for the upper surface of the cosmetic product cake which is to be obtained.

With a suitable dosing device 3 (FIG. 2), a dosed amount of cosmetic product 4, in powder or fluid state, is then introduced into the mold 1, possibly subjected to a slight pressure from above to form a layer of uniform height such as that shown in FIG. 3.

A further dosing device 5 (FIG. 4) then pours a suitable dose of the previously prepared aqueous fluid mixture 6 on the cosmetic product layer 4, where by virtue of the fluid state thereof, it spreads so as to form a substantially uniform fluid mixture layer 6.

The mold 1 with the cosmetic product layer 4 and the overlapping fluid mixture layer 6 is then placed in an oven at 60° C., where the drying of the aqueous part of the fluid layer 6 occurs along with the formation of a solidified set 8 (FIG. 5) consisting of a lower layer 4 of cosmetic product and an upper layer 6 consisting of a solidified mixture of natural ingredients.

The set 8 is finally overturned and introduced into a container 9 (FIG. 6), where the layer 4 of solidified cosmetic product with a shaped upper surface is supported and firmly anchored by means of a lower layer 6 (also referred to as a “grid”) of natural type, without plastic ingredients.

1-14. (canceled)

15. A cosmetic product support consisting of a solid flat layer formed by a dried and solidified mixture of water and additional ingredients, all said additional ingredients being of natural origin.

16. The cosmetic product support of claim 15, wherein said mixture has the following formulation:
a) water from 20.00% to 80.00% by weight;
b) organic acids from 0.05% to 2.00% by weight;
c) cellulose or derivatives from 1.00% to 10.00% by weight;
d) vegetal proteins from 0.10% to 20.00% by weight;
e) sugars from 1.00% to 20.00% by weight;
f) starches or dextrins from 10.00% to 70.00% by weight;
g) mica from 10.00% to 20.00% by weight; and
h) preservatives from 0.20% to 1.00% by weight.

17. The cosmetic product support of claim 15, wherein said mixture has the following formulation:
a) water in an amount of 59.20% by weight;
b) organic acids in an amount of 0.20% by weight;
c) cellulose or derivatives in an amount of 1.00% by weight;
d) vegetal proteins in an amount of 1.00% by weight;
e) sugars in an amount of 8.10% by weight;
f) starches or dextrins in an amount of 15.50% by weight;
g) mica in an amount of 14.00% by weight; and
h) preservatives in an amount of 1.00% by weight.

18. The cosmetic product support of claim 16, wherein said vegetal proteins are proteins of peas, lentils, beans, broad beans, and the like.

19. The cosmetic product support of claim 16, wherein said mixture comprises foamed hydrocolloids derived from cellulose, in particular crystalline cellulose, methylcellulose, hydroxypropyl cellulose, hydroxypropyl methylcellulose, carboxymethyl cellulose.

20. The cosmetic product support of claim 16, wherein said sugars are monosaccharide, disaccharide, or polysaccharide sugars of vegetal origin (maize, rice, tapioca, wheat, or legumes).

21. The cosmetic product support of claim 16, wherein said sugars are derived from enzymatically dextrinized starch.

22. The cosmetic product support of claim 15, wherein it is manufactured as a cosmetic wafer comprising the following ingredients:
a) non-allergenic but texturizing vegetable proteins
b) rice starches of the type used in lipsticks
c) maltodextrins and/or sugars
d) hydroxypropyl cellulose
e) cosmetic oil
f) hydrogenated lecithin powder
g) mica- and/or talc-based filler
h) hydrocolloids
i) water.

23. A process for forming a cosmetic product and support assembly, comprising the following steps:
a) preparing an aqueous fluid mixture of water and non-synthetic ingredients of natural origin,
b) introducing a dosed amount of cosmetic product into a mold for forming a cosmetic product layer overlying a bottom wall of said mold,
c) pouring a dosed amount of said aqueous fluid mixture into said mold above said cosmetic product layer to form an aqueous fluid mixture layer overlying said cosmetic product layer,
d) inserting said mold with said cosmetic product aqueous fluid mixture layers into a drying and solidifying ovento form a dried and solidified assembly,
e) extracting the mold from said oven,
h) overturning the mold,
i) extracting said dried and solidified assembly from the mold.

24. The process of claim 23, wherein said aqueous mixture has the following formulation:
a) water from 20.00% to 80.00% by weight;
b) organic acids from 0.05% to 2.00% by weight;
c) cellulose or derivatives from 1.00% to 10.00% by weight;
d) vegetal proteins from 0.10% to 20.00% by weight;
e) sugars from 1.00% to 20.00% by weight;
f) starches or dextrins from 10.00% to 70.00% by weight;
g) mica from 10.00% to 20.00% by weight; and
h) preservatives from 0.20% to 1.00% by weight.

25. The process of claim 23, characterized in that said aqueous mixture has the following formulation:
a) water in an amount of 59.20% by weight;
b) organic acids in an amount of 0.20% by weight;
c) cellulose or derivatives in an amount of 1.00% by weight;
d) vegetal proteins in an amount of 1.00% by weight;
e) sugars in an amount of 8.10% by weight;
f) starches or dextrins in an amount of 15.50% by weight;
g) mica in an amount of 14.00% by weight; and
h) preservatives in an amount of 1.00% by weight.
